(19) United States
(12) Patent Application Publication
(10) Pub. No.: US 2004/0116679 A1
(43) Pub. Date: Jun. 17, 2004

(54) Method and a plant for the separation of fat from proteins in whey materials

(76) Inventors: Preben Busch Kønigsfeldt, (US); Chris Burt, Melbourne (AU); Mark Allan Litchfield, Silkeborg (DK)

Correspondence Address: Cameron K Weiffenbach, McDermott Will & Emery, 600 13th Street N W, Washington, DC, 20005-3096, US

(21) Appl. No.: 10/474,065

(22) PCT Filed: Apr. 3, 2002
(86) PCT No.: PCT/DK02/00222

(30) Foreign Application Priority Data
Apr. 4, 2001 (DK) PA 2001 00554

(51) Int. Cl. A23J 1/00; A23C 17/00; A23C 21/00; C07K 1/34

(52) U.S. Cl. 530/414; 426/583

(57) ABSTRACT

A method and a plant for the separation of fat from proteins in a whey material to obtain a whey protein isolate (WPI) with a low fat content comprising the combination of a centrifugal separator and microfiltration (MF). The material fed to the separator contains the MF-retentate not passing through the microfilter. The combination improves the efficiency of the centrifugal separator and increases the yield of valuable WPI.

### TECHNICAL FIELD

The present invention relates to a method for the separation of fat from proteins in a whey material to obtain a whey protein isolate (WPI) with a low fat content as well as a plant to be used for carrying out the method.

### TECHNICAL BACKGROUND

Whey is a side product of conventional cheese preparation. It is the aqueous phase of milk that is separated from the coagulable part or curd especially in the process of making cheese. Typically whey forms about 80 to 90% by weight of the milk and about 50% of its nutritive content. It is rich in lactose, minerals and vitamins, and soluble proteins (known as whey proteins) and contains traces of fat. According to WO 93/21781 (Alfa-Laval) the dry solids content (TS) of whey is about 6.3% by weight of which 4.85% are lactose, 0.8% protein, 0.5% fat and 0.7% salts. However, the composition may vary depending on the cheese making process from which the whey originates.

Whey protein constituents include especially β-lactoglobulin and α-lactalbumin, but also immunoglobulines and bovine serum albumin.

Previously whey was considered as a problematic waste product without any value and it was in fact often discharged in lakes and water courses. Later on the whey was used as animal feed and as fertilizer. Today the whey is considered as a potential resource and efforts are being made to evaluate methods for separation and isolation of the different components in order to recover valuable and useful products.

Such valuable products includes whey protein concentrate (WPC). Preparation of WPC from a whey material normally involves a centrifugal separation reducing the fat content to typically about 3 to 8% by weight on dry matter basis.

WPC has a high nutritive value as well as important functional properties such as solubility, foaming and heat-set properties and emulsifying properties making WPC useful as an adjuvant in the food industry. However, in many cases such use as an adjuvant requires a very low fat content, which means that the above mentioned content of 3 to 8% by weight of fat would jeopardize the desired functional properties.

Therefore whey protein concentrates having a fat content below 1% by weight calculated on dry matter are specially valuable on the market, where they are termed whey protein isolates (WPI). The present market price as a dry powder product is 8 US $ per kg for WPI powder whereas the corresponding price for a WPC powder having a fat content of about 3 to 8% by weight calculated on dry matter is only 3 US $ per kg.

P. Logan, Dairy Technology, April 1991, pages 5 to 7 discloses a method of obtaining WPI by cross flow microfiltration of whey using a uniform low transmembrane pressure after centrifugal separation. The conditions for this appears in more details in WO 93/21781, which discloses a method of obtaining WPI by cross flow microfiltration of whey using a uniform low transmembrane pressure below 0.8×10⁵ Pa for removal of the major part of the remaining fat with the microfiltration retentate (MF-retentate). The MF-permeate, that is the fraction passing through the microfilter, is subjected to a further treatment giving a WPI defined as a whey protein product rich in β-lactoglobulin and α-lactalbumin but with a low fat content.

WO 93/21781 gives no information about any utilisation of the MF-retentate which apart from the fat and bacteria still contain a not negligible part of whey proteins. Further utilisation of this side product has been as butter oil which is a cheap fat containing product or by sending it back to the cheese milk. In both cases the high content of bacteria is a problem. In the case of butter oil a heat treatment is necessary and in the case of addition to the cheese milk the cheese making process can be disturbed or spoiled.

Due to the fact that a substantial part of the whey proteins contained in the untreated whey ends up in the MF-retentate the yield of whey proteins in the WPI end product obtainable according to prior art calculated according to the formula

$$yield in \% = \frac{proteins in the WPI}{proteins in the starting whey material} \times 100 \%$$

is normally from 65 to 80%.

Bearing in mind the very high value of WPI as compared with the lower value of WPC with a fat content of 3 to 8% by weight on dry matter basis there still exists a need for a better separation method whereby a higher amount of useful whey proteins could be isolated from a whey material while the content of fat in the isolated protein product would still fulfil the requirement of being below 1% by weight on dry matter basis.

EP 0 697 816 (APV Pasilac A/S) discloses a plant and a method for treating milk using a combination of a conventional separator and microfiltration where the material fed to the separator—milk—contains a MF retentate obtained by microfiltration of skimmed milk. The purpose of this recycling of the MF retentate is to avoid heat treatment of casein and other proteins remaining in the MF retentate. The separator used in EP 0 697 816 showed the same effect with respect to separation of cream as by normal use without addition of the recycled MF retentate.

DE 42 15 339 (Westfalia Separator AG) discloses a method for continuous preparation of a sterilised nutrient medium whereby the medium is microfiltrated and the retentate is centrifugated on a special bacteria removing centrifuge. The paper does not mention any fat content in the medium and accordingly it is silent about the efficiency of fat removal by the centrifuge.

### DESCRIPTION OF THE INVENTION

It has now been found that a special arrangement of a method using both a centrifugation step and a microfiltration step for the isolation of a whey protein isolate (WPI) leads to a better performance of the separator used in the centrifugation step giving a reduced loss of the valuable whey proteins and hence a WPI product in a higher yield based on the whey proteins contained in the starting whey material.

Accordingly the present invention relates to a method for the separation of fat from proteins in a whey material to obtain a whey protein isolate (WPI) with a low fat content comprising the combination of a centrifugation step using a separator separating the material fed thereto into a low density fraction, a high density fraction termed a skimmed fraction and optionally also a very high density fraction termed a sludge fraction and a microfiltration step (MF step) using a microfilter separating the material fed thereto into a MF-retentate retained by the microfilter and a MF-permeate passing through the microfilter whereby the material fed to the separator contains the MF-retentate obtained in the MF step.

It was surprisingly found that the effect of the separator is substantially improved when a MF retentate obtained by microfiltration of a whey material is added to the material fed to the separator. An improved effect in this connection is a good separation of the low density fraction from the remaining skimmed fraction with strongly reduced fat content.

Although addition of a MF retentate to the feed material to a separator per se is known from EP 0 697 816 such improved effect of the separator did not appear in that case where the material fed to the separator was milk.

The present invention also relates to a plant for the separation of fat from proteins in a whey material to obtain a whey protein isolate (WPI) with a low fat content comprising a centrifugation unit separating the material fed thereto into a low density fraction, a high density fraction termed a skimmed fraction and optionally also a very high density fraction termed a sludge fraction and a microfiltration unit (MF unit) with a microfilter separating the material fed thereto into a MF-retentate retained by the microfilter and a MF-permeate passing through the microfilter, wherein the MF unit and the centrifugation unit are connected with conduits conducting the MF-retentate obtained from the MF unit to the inlet of the centrifugation unit.

The extent of applicability of the invention appears from the following detailed description. It should, however, be understood that the detailed description and the specific examples are merely included to illustrate the preferred embodiments, and that various alterations and modifications within the scope of protection will be obvious to persons skilled in the art on the basis of the detailed description.

### BRIEF DESCRIPTION OF THE DRAWINGS

The invention is described in greater detail below with reference to the accompanying drawing and example. In the drawing

FIG. 1 is a schematic flow diagram of a plant according to claim 16 to be used when carrying out the method according to the embodiment of claim 4, and

FIG. 2 is a schematic flow diagram of a plant according to claim 17 to be used when carrying out the method according to the alternative embodiment of claim 6.

### BEST MODE FOR CARRYING OUT THE INVENTION

The present invention is based on a special combination of two per se usual separation methods:

A centrifugation step using a conventional separator which separates the material fed to the separator in fractions based in the density:

a low density fraction, which for practical reasons can be termed the “cream fraction” or just “cream” or, as the feed material is a whey material it can more correctly be termed “whey cream”,

a high density fraction from which the whey cream has been skimmed; this fraction is also called the skimmed fraction or skimmed whey,

furthermore a very high density fraction or sludge is separated by most of the conventional types of separators. The sludge fraction is only a small portion of the feed material. It contains inter alia a large portion of the bacteria and bacteria spores.

A MF step (microfiltration step) using a cross-flow MF unit which separates the material fed to the MF unit in two fractions based on penetration through the microfilter membrane:

a MF retentate which does not penetrate through the MF membrane, and

a MF permeate which passes through the MF membrane.

Both the centrifugation step, the MF step as well as combinations thereof are known per se but an essential new feature of the invention is that the material fed to the centrifugal separator contains a MF retentate obtained from MF of a whey material.

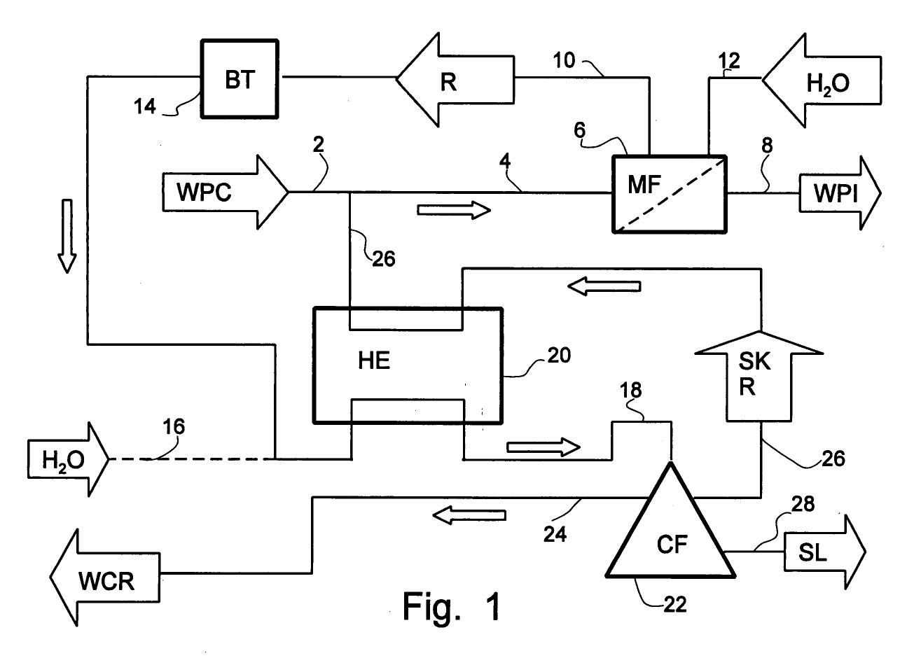

This essential feature can be established in different ways. Below a first embodiment shall be explained with reference to FIG. 1, using following abbreviations:

| | |
|---|---|
| WPC | Whey protein concentrate |
| WPI | Whey protein isolate |
| MF | Microfiltration unit |
| R | Retentate |
| BT | Buffer tank |
| HE | Heat exchanger |
| CF | Centrifugal separator |
| SK R | Skimmed retentate |
| SL | Sludge |
| WCR | Whey cream. |

The starting whey material to be treated by the inventive method according to the first embodiment is obtained from whey which has first been skimmed to remove the first major portion of the fat followed by preconcentraton by ultrafiltration (UF). This starting whey material is a whey protein concentrate obtained as the UF retentate (UF WPC).

As shown in FIG. 1 the WPC, which may be the UF WPC obtained as described above, is fed through a conduit 2 and 4 directly to a MF unit 6 where it is separated into the WPI product obtained in a conduit 8 as the MF permeate and the MF retentate obtained in a conduit 10. In case of diafiltration water can be added to the MF unit through a conduit 12. The MF retentate (R) may be lead to a buffer tank (BT) 14 and optionally the MF retentate can be diluted with water or available permeate streams from a conduit 16. Further the MF retentate passes in a conduit 18 through a heat exchanger (HE) 20 to a centrifugal separator (CF) 22. Here the MF retentate is separated into the light whey cream fraction (WCR) in conduit 24, a skimmed fraction (skimmed retentate; SK R) in conduit 26 and a minor sludge fraction (SL) in conduit 28. Then the skimmed retentate passes through the heat exchanger 20 whereafter it is mixed with the WPC from conduit 2 forming a mixture in conduit 4. This mixture is fed to the MF unit 6.

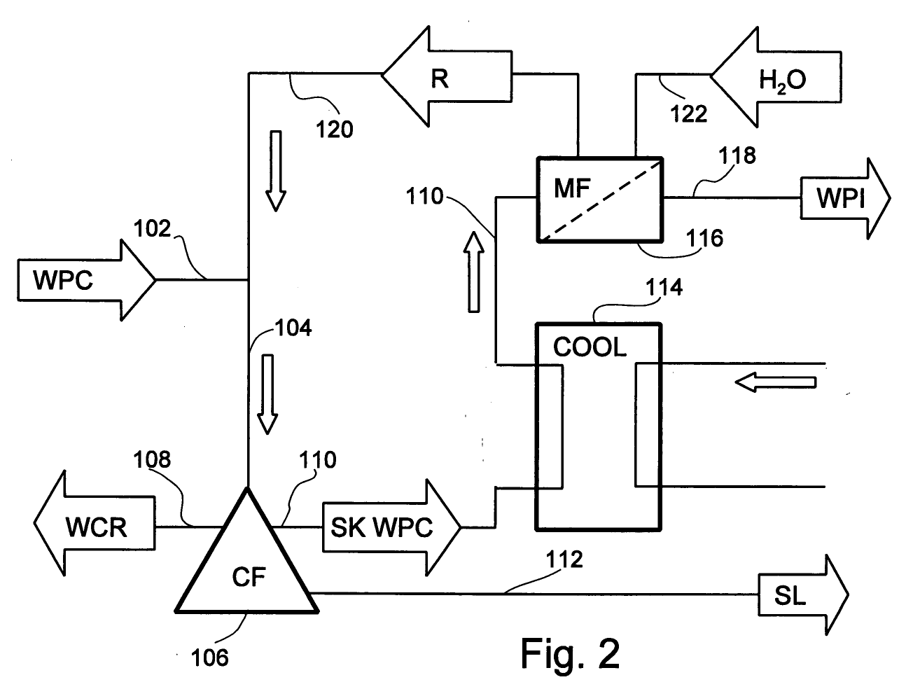

A second embodiment shall be explained with reference to FIG. 2 using the above abbreviations used in FIG. 1 and further

| | |
|---|---|
| SK WPC | Skimmed whey protein concentrate |
| COOL | Cooler |

The starting whey material to be treated by the inventive method according to the second embodiment is obtained from whey which has been preconcentrated by ultrafiltration (UF). This starting whey material is a whey protein concentrate obtained as the UF retentate (UF WPC).

As shown in FIG. 2 the WPC, which may be the UF WPC obtained from unskimmed whey as described above, is fed through a conduit 102 and 104 directly to a centrifugal separator (CF) 106. Here the WPC is separated into the light whey cream fraction (WCR) in conduit 108, a skimmed fraction (skimmed whey protein concentrate; SK WPC) in conduit 110 and a minor sludge fraction (SL) in conduit 112. The skimmed WPC passes in the conduit 110 through a cooler (COOL) 114 and further to a MF unit 116 where it is separated into the WPI product obtained in a conduit 118 as the MF permeate and the MF retentate (R) obtained in a conduit 120. In case of diafiltration water or membrane permeates can be added to the MF unit through a conduit 122. The MF retentate (R) is mixed with the WPC from conduit 102 forming a mixture in conduit 104. This mixture containing the MF retentate (R) is fed to the centrifugal separator (CF) 106.

In a third embodiment of the inventive method the starting material is unskimmed whey. Referring to FIG. 2 unskimmed whey is fed through the conduit 102 and 104 to the centrifugal separator (CF) 106. The skimmed fraction obtained in conduit 110 will then be skimmed whey in stead of skimmed whey protein concentrate (SK WPC). Apart from this the third embodiment comprises the same steps as the above illustrated second embodiment.

Centrifugation

The step of centrifugation can be carried out in a conventional separator whereby a fat containing fraction termed whey cream is skimmed off leaving a skimmed fraction having a low fat content. In principle the separator is of the same type as the conventional centrifugal separator used for the separation of milk into cream and skimmed milk or whey into whey cream and skimmed whey. Such separators will normally also separate a minor sludge fraction including a major part of the bacteria and spores being present. The sludge separation may be discontinuous but preferably it is continuous. An example of separators having continuous sludge separation is the so called self-cleaning separator centrifuges.

The centrifugation is usually carried out at 35 to 60° C., preferably at 45 to 55° C.

Microfiltration (MF)

The step of microfiltration can be carried out on a cross-flow microfiltration unit (MF unit) using a microfilter with an average pore size of 0.01 to 2 μm, preferably 0.05 to 0.8 μm, and most preferred 0.09 to 0.5 μm. Usually MF is carried out at 40 to 55° C., especially about 50° C. Thus useful results have been obtained by MF at 45 to 55° C. Surprisingly, however, excellent results was also found at 10 to 25° C. In fact, based on the test runs carried out it is believed that the best results are obtained when the MF step of the present invention is carried out cold such as below 35° C., preferably between 10 and 25° C.

In the MF step the material fed to the MF unit is concentrated with the concentration factor Fc=2 to 20, preferably 4 to 10, whereby

$$Fc = \frac{F}{R}$$

F=volume of feed material to the MF unit, and

R=volume of the retentate.

The MF permeate obtained in the MF step is a whey protein material with a very low fat content such as below 1% by weight. This is a high value product known as whey protein isolate (WPI).

The MF step is preferably carried out using the cross-flow principle in conventional microfiltration units which may be of differing structural shapes. As a basic model a microfiltration unit (MF unit) with cross flow may be formed of a container divided by a microfiltration membrane into two chambers, a feed/retentate chamber and a permeate chamber. The retentate chamber is provided with a feed conduit for feeding the material to be filtered, and a retentate outlet. The permeate chamber is provided with a permeate outlet. Between the retentate chamber and the permeate chamber a pressure difference is established forcing the fluid and small particles through the membrane. The feed material is fed through the retentate chamber from one side along the membrane. On the other side of the retentate chamber the retentate is removed, said retentate consisting of the fluid and the particles, which have not passed through the membrane to the permeate chamber during the passage along the membrane. In order to prevent the membrane surface from being fouled too quickly, which causes clogging of the membrane pores, the flow rate (cross-flow rate) over the surface of the membrane should not be too low. This is often ensured by recirculating a portion of the retentate flow to the feed conduit. It is also well-known to recirculate a portion of the permeate to ensure a uniform pressure drop, the permeate chamber in addition to the permeate outlet also being provided with an inlet for receiving recirculated permeate. This principle is described in U.S. Pat. No. 4,105,547 (Sandblom). Such recirculation conduits for retentate or permeate leading to the same respective retentate chamber or permeate chamber from which said material has flown, are considered as components forming part of a basic model of the microfiltration unit.

The MF unit used in the MF step according to the present invention can include one or more of such basic models. In case of more such basic models are used as the MF unit they can be coupled in different ways such as in series and/or parallel. Such couplings of several filtration membranes are well-known to persons skilled in the art. Examples of different coupling systems for membrane filtration are disclosed in WO 00/74495 (APV Pasilac A/S).

The MF unit used in the MF step may have any conventional shape. Examples hereof are any type selected from among the plate-and-frame system, a tubular system, a spiral-wound system, a cassette system and the hollow fibre principle or a combination thereof.

The membranes used in the MF unit can be made of various materials, especially ceramic or organic materials, such as aluminiumoxide, zirconium oxide, titanium oxide or mixtures thereof, polysulphones, fluoropolymers. The membrane is usually of a pore size in the range from 0.01 μm to 2 μm, preferably 0.05 to 0.8 μm, and most preferred 0.09 to 0.5 μm.

The whey material used as the starting material in the method according to the invention can be any whey material containing whey proteins. Preferably the starting whey material has been preconcentrated, for example by evaporation, reverse osmosis or ultrafiltration (UF), most preferred by UF.

Preferably the starting whey material has been preconcentrated to a 1 to 20 fold smaller volume.

Preconcentration by UF is preferably carried out with a UF filter having a molecular cutoff value of 500 to 50000 dalton, most preferred 1000 to 25000 dalton, using a concentration factor Fc of 1 to 20, preferably 2 to 10.

In case the starting whey material is conducted directly to the MF step as in the above mentioned first embodiment, it is preferred that the fat content thereof has been reduced by a pre-skimming process. This will reduce the risk of early clogging of the MF filter.

This pre-skimming process is preferably carried out before the preconcentration.

On the other hand, in case the starting whey material first is conducted to the centrifugation step as in the above mentioned second and third embodiments such pre-skimming process may be omitted.

The present invention is further illustrated by the following example, wherein % is by weight unless otherwise specified.

### EXAMPLE

Untreated whey was clarified and skimmed and the skimmed whey was concentrated by UF with a concentration factor Fc of 3.5 to obtain an ultrafiltrated whey protein concentrate (UF WPC) with a dry matter content (total solids; TS) of 11.4%; a total protein content (TOP) of 6.15% and a fat content of 0.40%. The remaining solids is 4.13% lactose, 0.17% acids, 0.48% soluble ash and 0.10% non-soluble ash.

This ultrafiltrated WPC was treated in the plant shown in FIG. 1. The average flow of WPC added through the conduit 2 was 16,826 kg/h. Supplementary water was added through the conduits 12 and 16. The resulting WPI obtained through the conduit 8 was in average 28,282 kg/h with 6.32% TS, 3.43% TOP and only 0.022% fat. The yield of TOP in the obtained WPI was 93.9%.

Still referring to FIG. 1 the flow and TS, TOP and fat in the conduits are shown in the table below:

| | | | TS | | |
|---|---|---|---|---|---|
| | Conduit | flow | % by | TOP | Fat |
| Material | | kg/h | weight | % by weight | % by weight |
| WPC | 2 | 16826 | 11.42 | 6.15 | 0.4 |
| SK R | 26 | 9169 | 7.68 | 6.35 | 0.25 |
| WPC + SK R | 4 | 25995 | 10.1 | 6.21 | 0.34 |
| Water | 12 | 6400 | 0 | 0 | 0 |
| R | 10 | 4113 | 20.39 | 15.7 | 2.02 |
| WPI | 8 | 28282 | 6.32 | 3.43 | 0.02 |
| Water | 16 | 6109 | 0 | 0 | 0 |
| WCR | 24 | 853 | 13.93 | 5.92 | 7 |
| SL | 28 | 200 | 7.68 | 6.35 | 0.25 |

The above description of the invention reveals that it is obvious that it can be varied in many ways. Such variations are not to be considered a deviation from the scope of the invention, and all such modifications which are obvious to persons skilled in the art are also to be considered comprised by the scope of the succeeding claims.

1. A method for the separation of fat from proteins in a whey material to obtain a whey protein isolate (WPI) with a low fat content comprising the combination of a centrifugation step using a separator separating the material fed thereto into a low density fraction, a high density fraction termed a skimmed fraction and optionally also a very high density fraction termed a sludge fraction and a microfiltration step (MF step) using a microfilter separating the material fed thereto into a MF-retentate retained by the microfilter and a MF-permeate passing through the microfilter whereby the material fed to the separator contains the MF-retentate obtained in the MF step.

2. A method as claimed in claim 1, wherein the starting whey material has been preconcentrated.

3. A method as claimed in claim 2, wherein the starting whey material has been preconcentrated by ultrafiltration (UF).

4. A method as claimed in any one of the preceding claims, wherein the skimmed fraction obtained in the centrifugation step is combined with the starting whey material forming a mixture, which mixture is the material fed to the microfilter.

5. A method as claimed in claim 4, wherein the starting whey material is preconcentrated skimmed whey.

6. A method as claimed in any one of the claims 1-3, wherein the skimmed fraction obtained in the centrifugation step is the material fed to the microfilter and wherein the MF-retentate is combined with the starting whey material forming a mixture, which mixture is the material fed to the separator.

7. A method as claimed in claim 6, wherein the starting whey material is preconcentrated unskimmed whey.

8. A method as claimed in any one of the preceding claims, wherein the MF step is carried out at 10-55° C.

9. A method as claimed in claim 8, wherein the MF step is carried out at 10-25° C.

10. A method as claimed in claim 2, wherein the starting whey material has been preconcentrated to a 1 to 20 fold smaller volume.

11. A method as claimed in any one of the claims 2 and 10 wherein the preconcetration has been carried out by UF using a UF filter having a molecular cut-off value of 1000 to 25000 dalton.

12. A method as claimed in claim 11, wherein the starting whey material has been preconcentrated by UF to a 2 to 10 fold smaller volume.

13. A method as claimed in claim 1, wherein the microfilter used in the MF step has a pore size of 0.05 to 0.8 μm.

14. A method as claimed in claim 1, wherein the MF step is carried out with a 4 to 10 fold concentraton rate based on the volume.

15. A plant for the separation of fat from proteins in a whey material to obtain a whey protein isolate (WPI) with a low fat content comprising
a centrifugation unit (22, 106) separating the material fed thereto into a low density fraction, a high density fraction termed a skimmed fraction and optionally also a very high density fraction termed a sludge fraction and
a microfiltration unit (MF unit) (6, 116) with a microfilter separating the material fed thereto into a MF-retentate retained by the microfilter and a MF-permeate passing through the microfilter,
whereby the MF unit and the centrifugation unit are connected with conduits (10, 18; 120, 104) conduiting the MF-retentate obtained from the MF unit to the inlet of the centrifugation unit.

16. A plant as claimed in claim 15, further comprising a conduit (26) for the skimmed fraction (SK R) obtained from the centrifugation unit (22) connected with an inlet conduit (2) for the feed whey material to be treated, which conduits (26 and 2) are joined in an MF inlet conduit (4) conducting the mixture of feed whey material and the skimmed fraction to the inlet of the MF unit (6).

17. A plant as claimed in claim 15, further comprising a conduit (120) for the retentate (R) obtained from the MF unit (116) connected with an inlet conduit (102) for the feed whey material to be treated, which conduits (120 and 102) are joined in an separator inlet conduit (104) conducting the mixture of feed whey material and the retentate to the inlet of the centrifugation unit (106).
